(19) United States
(12) Patent Application Publication
(10) Pub. No.: US 2012/0183500 A1
(43) Pub. Date: Jul. 19, 2012

(54) ARTICLES AND METHODS INCLUDING STABLE THERMALLY-RESPONSIVE POLYMERS

(75) Inventors: Emily Rose Rolfes Meyering, Eden Prairie, MN (US); Toni M. Heyer, St. Louis Park, MN (US)

(73) Assignee: SURMODICS, INC., Eden Prairie, MN (US)

(21) Appl. No.: 13/185,041

(22) Filed: Jul. 18, 2011

Related U.S. Application Data
(60) Provisional application No. 61/366,020, filed on Jul. 20, 2010.

(51) Int. Cl. A61K 47/32; B01D 57/02; A61K 38/02; A61K 31/7088; C08F 220/60; A61K 35/00

(52) U.S. Cl. 424/93.1; 514/772.4; 526/307.2; 526/307.3; 526/307.5; 514/1.1; 514/44 R; 204/456

(57) ABSTRACT

Embodiments of the invention can include or be used in conjunction with implantable medical devices and various in vitro applications. In an embodiment, an implantable medical device is included herein. The device can include a frame having an inner lumen; the frame comprising a copolymer formed from monomers comprising N-isopropylacrylamide or a thermally responsive derivative thereof and a second monomeric species including one or more of a heteroatom or a charged moiety. In an embodiment, a method of isolating a component of a mixture is included herein. The method can include mixing a sample with a copolymer formed from monomers comprising N-isopropylacrylamide or a thermally responsive derivative thereof and a second monomeric species including one or more of a heteroatom or a charged moiety to form a reagent mixture, the copolymer further comprising a moiety exhibiting affinity for an analyte in the sample. The method can further include raising the temperature of the reagent mixture to high enough for the copolymer to form a hydrogel. Other embodiments are also included herein.

This application claims the benefit of U.S. Provisional Application No. 61/366,020, filed Jul. 20, 2010, the contents of which are herein incorporated by reference.

### FIELD OF THE INVENTION

The present invention relates to the use of thermally responsive polymers for various applications. More specifically, the present invention relates to the use of these polymers at elevated temperatures.

### BACKGROUND OF THE INVENTION

Contrary to the behavior of most compounds in aqueous solutions, thermally responsive polymers become less soluble (more hydrophobic) in water at elevated temperatures. Poly(N-isopropylacrylamide) (poly-NIPAAm) is an exemplary thermally-responsive polymer. The point in which poly-NIPAAm undergoes a phase transition from soluble to insoluble (lower critical solution temperature or LCST) has been determined to occur at 32° C. at neutral pH without the presence of other compounds altering ionic strength.

The properties of NIPAAm above its LCST change from a swelled gel to a condensed gel as the temperature is raised. At a certain temperature, the gel starts to push out the water, causing the gel to fall out of solution and shrink.

### SUMMARY OF THE INVENTION

Embodiments of the invention relate to the use of thermally responsive polymers for various applications. In an embodiment, a method of implanting a medical device is included herein. The method can include injecting a copolymer at a first temperature into a subject, the copolymer formed from monomers comprising N-isopropylacrylamide or a thermally responsive derivative thereof and a second monomeric species including one or more of a heteroatom or a charged moiety. The method can further include forming the copolymer into a shape comprising an inner lumen, wherein the copolymer increases in temperature after residence in the subject to a second temperature and stiffens at the second temperature to maintain the inner lumen.

In an embodiment, an implantable medical device is included herein. The device can include a frame defining a central lumen; the frame comprising a copolymer formed from monomers comprising N-isopropylacrylamide or a thermally responsive derivative thereof and a second monomeric species including one or more of a heteroatom or a charged moiety.

In an embodiment, a method of providing a biological material substrate is included herein. The method can include mixing a biological material with a copolymer formed from monomers comprising N-isopropylacrylamide or a thermally responsive derivative thereof and a second monomeric species including one or more of a heteroatom or a charged moiety. The method can further include increasing the temperature of the copolymer to form a hydrogel and allowing the biological material to undergo activity at the increased temperature. The method can further include lowering the temperature of the copolymer until it dissolves. The method can further include separating the biological material from the copolymer.

In an embodiment, a biological material substrate is included herein. The biological material substrate can include a copolymer formed from monomers comprising N-isopropylacrylamide or a thermally responsive derivative thereof and a second monomeric species including one or more of a heteroatom or a charged moiety; and a biological material.

In an embodiment, a method of separating out components of a composition is included herein. The method can include forming a hydrogel comprising raising the temperature of a copolymer formed from monomers comprising N-isopropylacrylamide or a thermally responsive derivative thereof and a second monomeric species including one or more of a heteroatom or a charged moiety to form a reagent mixture. The method can further include passing a mixture to be separated through the hydrogel. The method can further include segmenting the hydrogel into two or more parts. The method can further include selecting a segmented part of the hydrogel including a desired component of the mixture to be separated. The method can further include cooling the segmented part of the hydrogel until the copolymer dissolves. The method can further include separating the dissolved copolymer from the desired component of the mixture to be separated.

In an embodiment, a method of isolating a component of a mixture is included herein. The method can include mixing a sample with a copolymer formed from monomers comprising N-isopropylacrylamide or a thermally responsive derivative thereof and a second monomeric species including one or more of a heteroatom or a charged moiety to form a reagent mixture, the copolymer further comprising a moiety exhibiting affinity for an analyte in the sample. The method can further include raising the temperature of the reagent mixture to high enough for the copolymer to form a hydrogel. The method can further include separating the hydrogel from the remainder of the reagent mixture. The method can further include lowering the temperature of the copolymer such that the hydrogel dissolves.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope of the present invention is defined by the appended claims and their legal equivalents.

### BRIEF DESCRIPTION OF THE FIGURES

The invention may be more completely understood in connection with the following drawings, in which:

FIG. 1 is a schematic diagram exhibiting distinctions between normal thermally responsive polymers and thermally responsive polymers included herein that can be used to form stable hydrogels.

FIG. 2 is a schematic diagram of an apparatus for inserting an implantable medical device in accordance with various embodiments herein.

FIG. 3 is a schematic diagram of an implantable medical device in accordance with an embodiment herein.

While the invention is susceptible to various modifications and alternative forms, specifics thereof have been shown by way of example and drawings, and will be described in detail. It should be understood, however, that the invention is not limited to the particular embodiments described. On the contrary, the intention is to cover modifications, equivalents, and alternatives falling within the spirit and scope of the invention.

### DETAILED DESCRIPTION OF THE INVENTION

The embodiments of the present invention described herein are not intended to be exhaustive or to limit the invention to the precise forms disclosed in the following detailed description. Rather, the embodiments are chosen and described so that others skilled in the art can appreciate and understand the principles and practices of the present invention.

All publications and patents mentioned herein are hereby incorporated by reference. The publications and patents disclosed herein are provided solely for their disclosure. Nothing herein is to be construed as an admission that the inventors are not entitled to antedate any publication and/or patent, including any publication and/or patent cited herein.

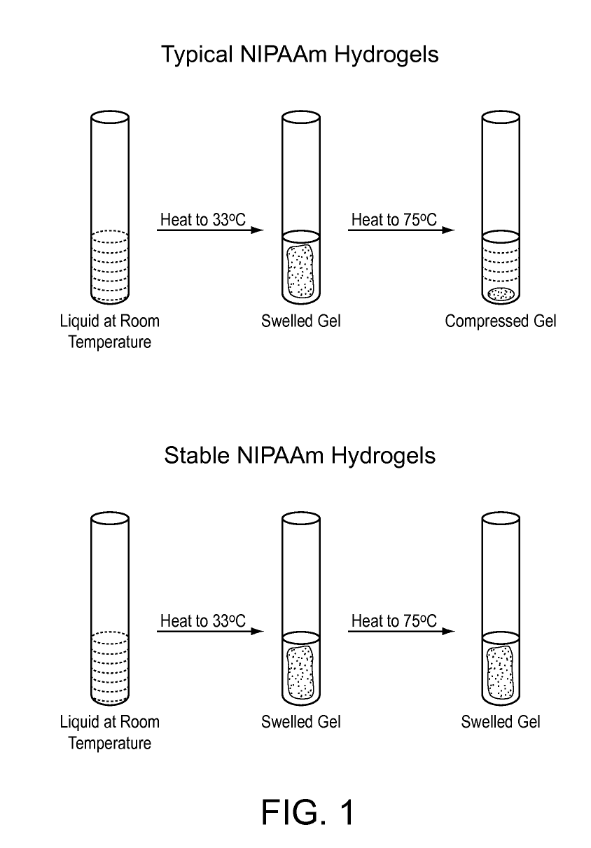

Thermally responsive polymers exhibit the remarkable property of becoming less soluble (more hydrophobic) in aqueous solutions at elevated temperatures. Thermally responsive polymers are described as having a lower critical solution temperature (LCST) at which they undergo a phase transition from soluble to insoluble. Referring now to FIG. 1, typical thermally responsive polymers such as poly(N-isopropylacrylamide) homopolymers form a swelled hydrogel at a point above LCST (illustrated here as 33 degrees Celsius) but then collapse into a compressed state at a higher temperature (illustrated here as 75 degrees Celsius).

However, as shown herein, co-polymers of poly(N-isopropylacrylamide) and other monomers can be formed which exist as hydrogels above their LCST and can exist stably without substantially collapsing (e.g., without expelling substantial solvent) over an extended temperature range above their LCST. As such, co-polymers included herein can be used to form hydrogels that are substantially stable over a temperature range above the LCST for the particular copolymer. Referring to FIG. 1, thermally responsive polymers included herein form a swelled hydrogel at a point above LCST (illustrated here as 33 degrees Celsius) and remain as a stable hydrogel at a higher temperature (illustrated here as 75 degrees Celsius) that would typically collapse normal thermally responsive polymers. It will be appreciated that the temperatures depicted in FIG. 1 are provided only for purposes of illustration and are not to be taken as limiting the scope herein.

Amongst other applications, in some embodiments, thermally responsive co-polymers as described herein can be used to form implantable medical devices. Specifically, thermally responsive co-polymers as described herein that can form hydrogels and stably maintain the hydrogel over a temperature range above gelation without substantially collapsing can be used to form implantable medical devices or parts thereof.

In some embodiments, thermally responsive co-polymers as described herein can be used to form implantable medical devices in situ. For example, some embodiments are directed to a method of implanting a medical device including injecting a copolymer at a first temperature into a subject. The copolymer can be formed from monomers comprising N-isopropylacrylamide or a thermally responsive derivative thereof and a second monomeric species including one or more of a heteroatom or a charged moiety. The method can also include forming the copolymer into a shape. It will be appreciated that various shapes are contemplated. For example, in the context of forming a stent in situ, the shape can include an inner lumen. After residence within the subject, the copolymer increases in temperature to a second temperature and can stiffen at the second temperature to maintain the desired shape. The second temperature can be above the LCST for the copolymer. For example, the copolymer can stiffen to maintain the inner lumen in the context of a stent.

As such, in an embodiment, an implantable medical device comprising a frame having an inner lumen is included. The frame comprising a copolymer formed from monomers comprising N-isopropylacrylamide or a thermally responsive derivative thereof and a second monomeric species including one or more of a heteroatom or a charged moiety.

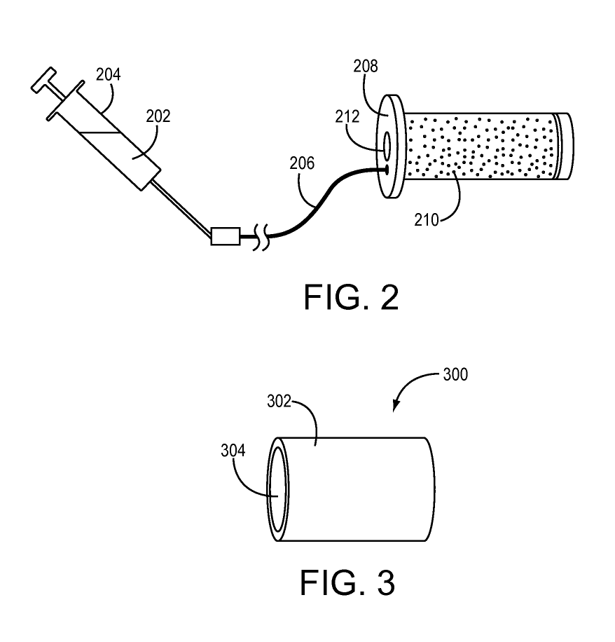

Referring now to FIG. 2, an apparatus is shown for inserting an implantable medical device in accordance with various embodiments herein. A solution 202 of the copolymer can be placed within an insertion device 204, such as a syringe. The solution 202 can then be injected through a catheter 206 to a shaping form 208. The shaping form 208 can include a plurality of pores 210 through which the solution 202 can pass. Once the solution passes out of the shaping form 208 it can contact the in vivo environment and the temperature of the solution can increase and the copolymer can stiffen. In some embodiments, the shaping form 208 is configured to be temporarily positioned within the lumen of a vascular tissue and can include a central lumen 212 to allow for the passage of bodily fluid during the insertion procedure. After the copolymer stiffens, thereby by forming an implantable medical device in situ, the shaping form 208 and catheter 206 can be withdrawn leaving behind the implantable medical device. Referring now to FIG. 3, a schematic diagram is shown of an implantable medical device 300 in accordance with an embodiment herein. The implantable medical device 300 can include a frame 302 defining a central lumen 304. The frame 302 can be relatively stiff resisting deformation and thereby keeping the central lumen open. In some embodiments, the frame 302 can be tubular in shape.

Beyond stents, it will be appreciated that many other implantable medical devices are included herein. By way of example, other medical devices including a copolymer as described herein can include, but are not limited to, vascular devices such as grafts (e.g., abdominal aortic aneurysm grafts, etc.), valves, embolic protection filters (including distal protection devices), aneurysm exclusion devices, mitral valve repair devices, vascular intervention devices, tissue scaffolds, orthopedic devices such as joint implants, acetabular cups, patellar buttons, bone repair/augmentation devices, spinal devices (e.g., vertebral disks and the like), cartilage repair devices, and artificial tendons; drug delivery devices such as injectable drug delivery depots and intravitreal drug delivery devices; ophthalmic devices including orbital implants, and intraocular lenses; urological devices and renal devices; and synthetic prostheses such as breast prostheses and artificial organs (e.g., pancreas, liver, lungs, heart, etc.), amongst others.

In addition to use with respect to implantable medical devices, it will be appreciated that copolymers as described herein have various in vitro applications. As one example, hydrogels formed with co-polymers as described herein can serve as substrates to support biological materials, such as supporting the growth of cells, or support biological components such as enzymes. In some embodiments, hydrogels can entrap the biological materials. In other embodiments, the hydrogels can serve as a surface onto with the biological materials are disposed or attached to. In some embodiments, hydrogels can mimic extracellular matrix in terms of interaction with cells and/or cellular components.

In such contexts, it will be appreciated that stability of a hydrogel over a range of temperatures above the point of gelation can facilitate various experimental procedures. As merely one example, it will be appreciated that induction of expression of heat shock proteins generally requires exposure to elevated temperatures. Such elevated temperatures may be sufficient to cause non-stable hydrogels formed with thermally responsive polymers to collapse. However, copolymers included herein can result in hydrogels that are stable at temperatures needed for induction of expression of heat shock proteins.

As such, in some embodiments, a method of providing a biological material substrate including mixing a biological material with a copolymer formed from monomers comprising N-isopropylacrylamide or a thermally responsive derivative thereof and a second monomeric species including one or more of a heteroatom or a charged moiety. The method can further include increasing the temperature of the copolymer to form a hydrogel and allowing the biological material to undergo activity at the increased temperature. In various embodiments, this can include increasing the temperature to a point above the LCST for the copolymer. It will be appreciated that the activity may be various things depending upon the particular assay considered. In some embodiments, the activity can include, but is not limited to, expression of a heat shock protein. The method can further include lowering the temperature of the copolymer until the hydrogel dissolves. In some embodiments, this can include lowering the temperature of the copolymer below the LCST for the copolymer.

In some embodiments the method can further include separating the biological material from the copolymer. It will be appreciated that separation can include many different possible techniques depending on physical and chemical distinctions between the copolymers used to form the hydrogel and the materials to be separated from the dissolved copolymers. By way of example, separation techniques can include but are not limited to, various types of chromatography such as size exclusion chromatography, centrifugation techniques, various forms of phase separation techniques, and the like. As another example of in vitro applications, copolymers included herein can be used to form hydrogels that can be used in techniques such as gel electrophoresis. By way of example, in an embodiment, a method of separating out components of a composition is included. The method can include forming a hydrogel comprising raising the temperature of a copolymer formed from monomers comprising N-isopropylacrylamide or a thermally responsive derivative thereof and a second monomeric species including one or more of a heteroatom or a charged moiety to form a reagent mixture.

The method can further include passing a mixture to be separated through the hydrogel. It will be appreciated that this can be accomplished in various ways. In one example, a sample or samples can be placed in individual wells formed near one edge of the formed hydrogel. An electrical field can be applied to the hydrogel. For example, a voltage differential can be created between the sides of the hydrogel causing components in the mixture to be separated to migrate through the hydrogel to the other side, separating out in terms of distance moved in the process based properties such as charge, size, and the like.

In some embodiments, the separation can be effectively tuned as it is believed that pore sizes within the can vary based on temperature. For example, in some embodiments, the pore size can decrease with increasing temperature over certain temperature ranges. It is believed that copolymers forming stable hydrogels as described herein can be advantageous in that the temperature can be varied to manipulate pore size without causing the hydrogel to collapse.

In some embodiments, after separation within the hydrogel, the results can be captured by, for example, labeling the components with an agent that is radio-labeled, or visualized through application of UV-light, or the like. However, in other embodiments, it may be desirable to physically isolate certain components for further assay steps. In this regard, in some embodiments, the method can further include physically segmenting the hydrogel into two or more parts. For example, a portion of the hydrogel containing a component of interest could physically be cut away and removed from the remainder of the hydrogel and other components. As such, in some embodiments, the method can further include selecting a segmented part of the hydrogel including a desired component of the mixture to be separated.

After a portion of the hydrogel is selected and physically isolated, then the component can be extracted by dissolving the hydrogel. As such, in some embodiments, the method can further include cooling the segmented part of the hydrogel until the copolymer dissolves. The method can further include separating the dissolved copolymer from the desired component of the mixture to be separated. As described above, it will be appreciated that separation can include many different possible techniques depending on physical and chemical distinctions between the copolymers used to form the hydrogel and the materials to be separated from the dissolved copolymers.

As another example of an exemplary in vitro application, stable hydrogels formed with co-polymers described herein can be used in techniques akin to affinity separation. In an embodiment, a method of isolating a component of a mixture is included. The method can include mixing a sample with a copolymer formed from monomers comprising N-isopropylacrylamide or a thermally responsive derivative thereof and a second monomeric species including one or more of a heteroatom or a charged moiety to form a reagent mixture. The copolymer can be modified to include a moiety exhibiting affinity for an analyte in the sample.

It will be appreciated that the moiety exhibiting affinity for an analyte in the sample can take on various forms depending on the nature of the desired analyte as well as the desired specificity of isolation amongst other factors. In some embodiments, the moiety can include polypeptides. By way of example, in some embodiments, the moiety can include an antibody or a fragment thereof. In some embodiments, the moiety can include a nucleic acid such as a polynucleotide that is at least partly complementary to an analyte of interest or a portion of an analyte of interest. As yet another example, it is known that maleimides react with thiol groups to form a bond. Utilizing this reaction, the moiety exhibiting affinity can be a maleimide group and thus the copolymer can be used to isolate proteins with a free thiol group. Many other moieties are contemplated within the scope herein.

The method can further include raising the temperature of the reagent mixture to high enough for the copolymer to form a hydrogel. In some embodiments, such as described above, the mixture to be separated can be combined with the copolymer forming the hydrogel prior to formation of the hydrogel (i.e., before raising the temperature of the reagent mixture). In such an embodiment, the formation of the hydrogel can effectively serve to precipitate out the co-polymer along with the desired analyte in the mixture

However, in other embodiments, the hydrogel can be formed first and then the mixture to be separated can be combined with the hydrogel. For example, the hydrogel may be formed and then the mixture can be passed through such as with a separation column.

In any case, after allowing sufficient time for the desired analyte to interact with the moiety exhibiting affinity for analyte, the other components of the original mixture can be separated out. As such, in some embodiments, the method can further include separating the hydrogel from the remainder of the reagent mixture. This can be done though various techniques such as centrifugation, filtration, and the like.

In some embodiments, such as where the hydrogel has bound with the analyte of interest, the method can further include lowering the temperature of the copolymer such that the hydrogel dissolves. For example, the method can include lowering the temperature of the hydrogel to a point below the LCST of the co-polymer used to form the hydrogel. Finally, in some embodiments, the co-polymer can be separated from the analyte of interest. It will be appreciated that it will be appreciated that separation can include many different possible techniques depending on the nature of the interaction of the moiety having affinity for the analyte of interest and the analyte, the physical and chemical distinctions between the copolymer used to form the hydrogel and the analyte of interest, and the like.

Autoclaving involves the application of high pressure steam at temperatures exceeding 100 degrees Celsius for a period of time in order to sterilize equipment and supplies. These conditions can affect many different types of materials. For example, autoclave conditions can be sufficient to cause hydrogels formed with normal thermally responsive polymers to collapse. However, in some embodiments, copolymers included herein can be used to form hydrogels that can be included in articles that remain stable while exposed to autoclave conditions. For example, in some embodiments, an article can be included that is configured to be exposed to autoclave conditions wherein the article comprises a copolymer as described herein.

### Thermally Responsive Copolymers Forming Stable Hydrogels

Embodiments herein can include thermally responsive copolymers that form stable hydrogels. Exemplary thermally responsive copolymers can include those that form a hydrogel at a given temperature and exhibit less than or equal to 25 percent volumetric shrinkage over a temperature range of at least about 10 degrees Celsius above the point at which they form a hydrogel. For example, thermally responsive copolymers can include those that form a hydrogel and exhibit less than or equal to 25 percent volumetric shrinkage over a temperature range of at least about 10 degrees Celsius above their LCST. In some embodiments, exemplary thermally responsive copolymers can include those that form a hydrogel an exhibit less than or equal to about 25 percent volumetric shrinkage over a temperature range of at least about 15 degrees above the point at which they form a hydrogel. In some embodiments, exemplary thermally responsive copolymers can include those that form a hydrogel an exhibit less than or equal to about 25 percent volumetric shrinkage over a temperature range of at least about 20 degrees above the point at which they form a hydrogel. In some embodiments, exemplary thermally responsive copolymers can include those that form a hydrogel an exhibit less than or equal to about 25 percent volumetric shrinkage over a temperature range of at least about 25 degrees above the point at which they form a hydrogel.

In some embodiments, exemplary thermally responsive copolymers remain as a stable hydrogel above 45 degrees Celsius. In some embodiments, exemplary thermally responsive copolymers remain as a stable hydrogel above 55 degrees Celsius. In some embodiments, exemplary thermally responsive copolymers remain as a stable hydrogel above 65 degrees Celsius. In some embodiments, exemplary thermally responsive copolymers remain as a stable hydrogel above 75 degrees Celsius. In some embodiments, exemplary thermally responsive copolymers remain as a stable hydrogel above 85 degrees Celsius. In some embodiments, exemplary thermally responsive copolymers remain as a stable hydrogel above 95 degrees Celsius. In some embodiments, exemplary thermally responsive copolymers remain as a stable hydrogel up to and above 100 degrees Celsius.

Exemplary thermally responsive polymers can include co-polymers derived from N-isopropylacrylamide and one or more other monomeric species. In some embodiments, thermally responsive polymers can include co-polymers derived from N-isopropylacrylamide and one other monomeric species. In some embodiments, thermally responsive polymers can include co-polymers derived from N-isopropylacrylamide and two other monomeric species.

It will be appreciated that the other monomeric species can be chose to impart specific characteristics to the thermally responsive polymer. In some embodiments, a monomeric species can be used that can electrostatically interface with water, such as through hydrogen bonding. In some embodiments, a monomeric species can be used including a heteroatom, such as a terminal heteroatom. In some embodiments, a monomeric species can be used including a charged moiety. In some embodiments, a monomeric species can be used including a quaternary amine. In some embodiments, a monomeric species can be used including one or more functional groups selected from thiol, amine, hydroxy, phosphate, sulphonate, and the like. In some embodiments, the monomeric species can include at least one of N,N-dimethylacrylamide (DMA), 2-hydroxyethyl methacrylate (HEMA), aminopropylmethacrylate hydrochloride (APMA), and 3-methacryloylaminopropyl trimethylammonium chloride (MAPTAC), or derivatives thereof. In an embodiment, the thermally responsive polymer can include co-polymers including monomer A and monomer B, wherein A is NIPAAm or a thermally responsive derivative thereof and B is monomer including one or more heteroatoms or charged moieties.

Relative proportions of NIPAAm versus other monomeric species present can depend on the properties desired. In some embodiments, exemplary thermally responsive co-polymers can include from about 60 to about 99 mole % NIPAAm and from about 1 to about 40 mole % of another monomeric species such as those described above. In some embodiments, exemplary thermally responsive co-polymers can include from about 70 to about 99 mole % NIPAAm and from about 1 to about 30 mole % of another monomeric species such as those described above. In some embodiments, exemplary thermally responsive co-polymers can include from about 75 to about 99 mole % NIPAAm and from about 1 to about 25 mole % of another monomeric species such as those described above. In some embodiments, exemplary thermally responsive co-polymers can include from about 80 to about 99 mole % NIPAAm and from about 1 to about 20 mole % of another monomeric species such as those described above. In some embodiments, exemplary thermally responsive co-polymers can include from about 85 to about 99 mole % NIPAAm and from about 1 to about 15 mole % of another monomeric species such as those described above. In some embodiments, exemplary thermally responsive co-polymers can include from about 90 to about 99 mole % NIPAAm and from about 1 to about 10 mole % of another monomeric species such as those described above. In some embodiments, exemplary thermally responsive co-polymers can include from about 95 to about 99 mole % NIPAAm and from about 1 to about 5 mole % of another monomeric species such as those described above.

Molecular weights of exemplary co-polymers can vary depending on the characteristics desired. In some embodiments, thermally responsive co-polymers include, but are not limited to, those with average molecular weights of about 1,000 Da to about 25,000 Da. In some embodiments, thermally responsive co-polymers include, but are not limited to, those with average molecular weights of about 3,000 Da to about 17,000 Da.

It will be appreciated that there are various techniques that can be used for forming exemplary co-polymers, depending on the specific monomer or monomers forming the co-polymer with poly-NIPAAm and the functional groups they include. By way of example, free radical chain polymerization is one technique that can be used. It will be appreciated that there are many free radical initiators known in the art that can be used, including but not limited to azo compounds such as 2-2′-azo-bis-isobutyrylnitrile (AIBN), organic peroxides such as benzoyl peroxide, metal iodides, metal alkyls, redox reagents, persulfates such as ammonium persulfate, and the like.

### Further Embodiments

In an embodiment, a method of implanting a medical device is included herein. The method can include injecting a copolymer at a first temperature into a subject, the copolymer formed from monomers comprising N-isopropylacrylamide or a thermally responsive derivative thereof and a second monomeric species including one or more of a heteroatom or a charged moiety. The method can further include forming the copolymer into a shape comprising an inner lumen, wherein the copolymer increases in temperature after residence in the subject to a second temperature and stiffens at the second temperature to maintain the inner lumen.

In an embodiment, the copolymer can include a substantially stable hydrogel from its lower critical solution temperature (LCST) to about 25 degrees Celsius above its LCST. In an embodiment, the second monomeric species can include at least one of N,N-dimethylacrylamide, 2-hydroxyethyl methacrylate, aminopropylmethacrylate hydrochloride, and 3-methacryloylaminopropyl trimethylammonium chloride. In an embodiment, the copolymer can include at least about 80 mole percent N-isopropylacrylamide. In an embodiment, the copolymer can have a molecular weight of about of about 3,000 Da to about 17,000 Da.

In an embodiment, an implantable medical device is included herein. The implantable medical device can include a frame defining a central lumen. The frame can include a copolymer formed from monomers comprising N-isopropylacrylamide or a thermally responsive derivative thereof and a second monomeric species including one or more of a heteroatom or a charged moiety. In an embodiment, the copolymer can include a substantially stable hydrogel from its lower critical solution temperature (LCST) to about 25 degrees Celsius above its LCST. In an embodiment, the second monomeric species can include at least one of N,N-dimethylacrylamide, 2-hydroxyethyl methacrylate, aminopropylmethacrylate hydrochloride, and 3-methacryloylaminopropyl trimethylammonium chloride. In an embodiment, the copolymer can include at least about 80 mole percent N-isopropylacrylamide. In an embodiment, the copolymer having a molecular weight of about of about 3,000 Da to about 17,000 Da.

In an embodiment, a method of providing a biological material substrate is included herein. The method can include mixing a biological material with a copolymer formed from monomers comprising N-isopropylacrylamide or a thermally responsive derivative thereof and a second monomeric species including one or more of a heteroatom or a charged moiety. The method can further include increasing the temperature of the copolymer to form a hydrogel and allowing the biological material to undergo activity at the increased temperature. The method can further include lowering the temperature of the copolymer until the hydrogel dissolves. The method can further include separating the biological material from the copolymer. In an embodiment, the biological material can comprise cells. In an embodiment, the biological material can comprise protein. In an embodiment, the biological material can comprise nucleic acid. In an embodiment, the hydrogel is configured to simulate extracellular matrix. In an embodiment, separating the biological material from the copolymer comprises size exclusion chromatography. In an embodiment, the copolymer can be a substantially stable hydrogel from its lower critical solution temperature (LCST) to about 25 degrees Celsius above its LCST. In an embodiment, the second monomeric species can include at least one of N,N-dimethylacrylamide, 2-hydroxyethyl methacrylate, aminopropylmethacrylate hydrochloride, and 3-methacryloylaminopropyl trimethylammonium chloride. In an embodiment, the copolymer can include at least about 80 mole percent N-isopropylacrylamide. In an embodiment, the copolymer can have a molecular weight of about of about 3,000 Da to about 17,000 Da.

In an embodiment, a biological material substrate is included herein. The biological material substrate can include a copolymer formed from monomers comprising N-isopropylacrylamide or a thermally responsive derivative thereof and a second monomeric species including one or more of a heteroatom or a charged moiety; and a biological material.

In an embodiment, a method of separating out components of a composition is included. The method can include forming a hydrogel comprising raising the temperature of a copolymer formed from monomers comprising N-isopropylacrylamide or a thermally responsive derivative thereof and a second monomeric species including one or more of a heteroatom or a charged moiety to form a reagent mixture; passing a mixture to be separated through the hydrogel; segmenting the hydrogel into two or more parts; selecting a segmented part of the hydrogel including a desired component of the mixture to be separated; cooling the segmented part of the hydrogel until the copolymer dissolves; and separating the dissolved copolymer from the desired component of the mixture to be separated.

In an embodiment, the method can further include adjusting the size of pores within the hydrogel by manipulating the temperature of the copolymer. In an embodiment passing the mixture to be separated through the hydrogel comprises creating a voltage differential between opposite sides of the hydrogel.

In an embodiment, a method of isolating a component of a mixture is included. The method can include mixing a sample with a copolymer formed from monomers comprising N-isopropylacrylamide or a thermally responsive derivative thereof and a second monomeric species including one or more of a heteroatom or a charged moiety to form a reagent mixture, the copolymer further comprising a moiety exhibiting affinity for an analyte in the sample; raising the temperature of the reagent mixture to high enough for the copolymer to form a hydrogel; separating the hydrogel from the remainder of the reagent mixture; and lowering the temperature of the copolymer such that the hydrogel dissolves. In an embodiment, the moiety exhibiting affinity for an analyte in the sample is configured to selectively form a complex with the analyte in the sample. In an embodiment, the moiety exhibiting affinity for an analyte in the sample comprises a polypeptide. In an embodiment, the moiety exhibiting affinity for an analyte in the sample comprises an antibody or a fragment thereof. In an embodiment, the moiety exhibiting affinity for an analyte in the sample comprises a polynucleotide. In an embodiment, the moiety exhibiting affinity for an analyte in the sample comprises a maleimide.

The present invention may be better understood with reference to the following examples. These examples are intended to be representative of specific embodiments of the invention, and are not intended as limiting the scope of the invention.

### EXAMPLES

### Example 1

### Formation of Thermally Responsive Polymers for Stable Hydrogels

Various copolymers of N-isopropylacrylamide were formed and tested for stability.

For samples 1-2, the procedure was as follows. Into a round-bottom flask, the polyethyleneglycol monomethacrylate (PEGMA) was dissolved into isopropyl alcohol and heated to 60° C. on a heating mantle with magnetic stirring. The flask was purged with nitrogen. The N-isopropylacrylamide (NIPAM) was also dissolved into isopropyl alcohol and the solution was placed into a liquid addition funnel with 2,2′-Azobis(2-methylpropionitrile) (AIBN). This was slowly added dropwise over one hour to the reaction flask at 60° C. The reaction proceeded overnight. The material was subsequently placed into 12-14 k MWCO dialysis tubing and was purified against deionized water for 48 hours. The product solution was lyophilized to obtain a white powder.

For samples 3-24, the procedure was as follows. Into an amber reaction vessel, all monomers were weighed and added. The reagents were dissolved in the appropriate solvent listed below and magnetically stirred at room temperature. Each vessel was purged by nitrogen bubbled through the solution for five minutes. Approximately 200 uL of the oxygen scavenger, N,N,N′,N′-tetrametylethylenediamine (TEMED) was added to each flask. The polymerization initiator (either AIBN or 10 wt % APS in water) was added to the flask with a final nitrogen purge and the vessel was sealed. The reactions were allowed to heat at 50° C. for 5-10 minutes to initiate polymerization but then remained stirring magnetically for 2-3 hours. Purification of the products was achieved by placing the crude reaction solution into 1 k or 12-14 k MWCO dialysis tubing in a constant flow deionized water dialysis tank. The dialysis proceeded for 48 hours and the final solutions were placed into trays and lyophilized until dryness to white powders.

The polymers listed above were dissolved in water and PBS buffer at 2.5, 5, or 10 weight % solids. Once they were fully dissolved, they were pipetted into small vials and placed into a heating oven. A temperature probe was added to the vials and the temperature was monitored as the samples heated. The cloud point (crude LCST) was determined when the solution became cloudy and viscous. At that point, the samples were compressed with a plastic rod to evaluate their physical properties. As the temperature of the vials continued to increase, and the gels formed, their physical properties were evaluated at 60° C. and documented. Several of the gels collapsed and were submerged in the buffer they had previously swelled. Samples that maintained their gelled state were heated with a heat gun to 100° C. and the physical properties were evaluated once again.

TABLE 1

| | Monomers | | | | | |
|---|---|---|---|---|---|---|
| | A | B | C | Molar Ratio | Initiator | Solvent |
| 1^{a} | NIPAM | PEG-MA_{375 M.W.} | — | 80:20:0 | AIBN | Isopropyl Alcohol |
| 2^{a} | NIPAM | PEG-MA_{1000 M.W.} | — | 90:10:0 | AIBN | Isopropyl Alcohol |
| 3^{a} | NIPAM | N-vinylpyrrolidone | — | 95:5:0 | APS | Water |
| 4^{a} | NIPAM | N-vinylpyrrolidone | — | 85:15:0 | APS | Water |
| 5^{a} | NIPAM | N-vinylpyrrolidone | — | 75:25:0 | APS | Water |
| 6^{a} | NIPAM | DMA | — | 90:10:0 | APS | Water |
| 7^{a} | NIPAM | DMA | — | 80:20:0 | APS | Water |
| 8^{a}* | NIPAM | DMA | — | 70:30:0 | APS | Water |
| 9^{a}* | — | DMA | MMA | 0:50:50 | AIBN | EtOH/Water |
| 10^{a}* | — | DMA | MMA | 0:40:60 | AIBN | EtOH/Water |
| 11^{a} | NIPAM | HEMA | APMA | 90:5:5 | APS | Water |
| 12^{a}* | Nt-BAM | N-vinylpyrrolidone | — | 95:5:0 | AIBN | EtOH/Water |
| 13^{a}* | Nt-BAM | N-vinylpyrrolidone | — | 85:15:0 | AIBN | EtOH/Water |
| 14^{a}* | Nt-BAM | N-vinylpyrrolidone | — | 75:25:0 | AIBN | EtOH/Water |
| 15^{a}* | Nt-BAM | HEMA | APMA | 90:5:5 | APS | Water |
| 16^{a}* | Nt-BAM | PEG-MA_{375 M.W.} | — | 80:20:0 | AIBN | Isopropyl Alcohol |
| 17^{a}* | Nt-BAM | PEG-MA_{375 M.W.} | — | 90:10:0 | AIBN | Isopropyl Alcohol |
| 18^{b} | NIPAM | APMA | — | 90:10:0 | APS | Water |
| 19^{b} | NIPAM | Ethyl Betaine | — | 90:10:0 | APS | Water |
| 20^{b} | NIPAM | Propyl Betaine | — | 90:10:0 | APS | Water |
| 21^{b} | NIPAM | HEMA | — | 90:10:0 | APS | Water |
| 22^{b} | NIPAM | HEMA | MAPTAC | 90:5:5 | APS | Water |
| 23^{b} | NIPAM | MAPTAC | — | 95:5:0 | APS | Water |
| 24^{b} | NIPAM | MAPTAC | MAA | 90:5:5 | APS | Water |

*Polymers crashed out of water during dialysis and were not further tested.
^{a}Polymers were dialyzed in 12-14k MWCO dialysis tubing.
^{b}Polymers were dialyzed in 1k MWCO dialysis tubing.
Nt-BAM (N-tertiary butylacrylamide); NIPAM (N-isopropylacrylamide); PEG-MA (polyethyleneglycol monomethacrylate); DMA (N,N-dimethylacrylamide); HEMA (2-hydroxyethyl methacrylate); APMA (aminopropylmethacrylate hydrochloride); MAPTAC (3-methacryloylaminopropyl trimethylammonium chloride).

TABLE 2

| | Solubility | | | |
|---|---|---|---|---|
| | 5 wt % | 10 wt % | Observations | Shrinkage at 60° C. |
| 1 | Y | Y | Did not gel | — |
| 2 | Y | Y | Did not gel | — |
| 3 | Y | Y | Soft gel at 35° C. | Collapsed |
| 4 | Y | Y | Hazy at 34° C. but no gel | — |
| 5 | Y | Y | Did not gel | — |
| 6 | Y | N | LCST ~35° C. | <25% |
| 7 | Y | Y | LCST ~35° C. | <25% |
| 11 | Y | N | LCST ~35° C. | None |
| 18 | Y | Y | Did not gel | — |
| 19 | Y | Y | Did not gel | — |
| 20 | Y | Y | Did not gel | — |
| 21 | Y | Y | LCST ~32° C. | None |
| 22 | Y | Y | LCST ~40° C. | None |
| 23 | Y | Y | LCST ~36° C. | None |
| 24 | Y | Y | Did not gel | — |

### Example 2

### Purification of Proteins Using Thermally Responsive Polymers

A thermoresponsive polymer is synthesized following the protocols listed for for samples 1-24 in Example 1 above, but also including an acrylated or methacrylated moiety containing a crosslinkable group such as a maleimide. After purification, this polymer is incubated with a solution containing a protein with a free thiol group. In neutral conditions at room temperature, the protein and polymer will react, leaving behind everything that does not contain a thiol group. The solution is then heated to a point above the LCST of the polymer in order to precipitate it from the solution. Next, the precipate hydrogel is rinsed with warm solution. After the particles are rinsed, they may be filtered or centrifuged out in order for collection. Then, the temperature is reduced and the polymer dissolves. Thus, once the temperature is reduced, the hydrogels become liquid. The polymer is then cleaved from the isolated protein.

### Example 3

### Stable Hydrogel for Evaluation of Heat Shock Proteins

In a cell culture plate, a solution of cells capable of expressing heat shock protein are mixed into an aqueous solution of a thermally responsive polymer. After the two solutions are adequately mixed, the temperature is raised and the cells are evenly dispersed and potentially attached to the hydrogel. At this raised temperature, the cells begin to express the heat shock proteins. After a sufficient time, the hydrogels in the plates are cooled dissolving the hydrogel and protein is extracted after processing steps such as cell lysis to test the expression levels between the conditions tested.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration to. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

The invention has been described with reference to various specific and preferred embodiments and techniques. However, it should be understood that many variations and modifications may be made while remaining within the spirit and scope of the invention.

1. A method of implanting a medical device comprising:
injecting a copolymer at a first temperature into a subject, the copolymer formed from monomers comprising N-isopropylacrylamide or a thermally responsive derivative thereof and a second monomeric species including one or more of a heteroatom or a charged moiety,
forming the copolymer into a shape comprising an inner lumen, wherein the copolymer increases in temperature after residence in the subject to a second temperature and stiffens at the second temperature to maintain the inner lumen.

2. The method of claim 1, the copolymer comprising a substantially stable hydrogel from its lower critical solution temperature (LCST) to about 25 degrees Celsius above its LCST.

3. The method of claim 1, the second monomeric species including at least one of N,N-dimethylacrylamide, 2-hydroxyethyl methacrylate, aminopropylmethacrylate hydrochloride, and 3-methacryloylaminopropyl trimethylammonium chloride.

4. The method of claim 1, the copolymer comprising at least about 80 mole percent N-isopropylacrylamide.

5. The method of claim 1, the copolymer having a molecular weight of about of about 3,000 Da to about 17,000 Da.

6. An implantable medical device comprising
a frame defining a central lumen; the frame comprising
a copolymer formed from monomers comprising N-isopropylacrylamide or a thermally responsive derivative thereof and a second monomeric species including one or more of a heteroatom or a charged moiety.

7. The implantable medical device of claim 1, the copolymer comprising a substantially stable hydrogel from its lower critical solution temperature (LCST) to about 25 degrees Celsius above its LCST.

8. The implantable medical device of claim 1, the second monomeric species including at least one of N,N-dimethylacrylamide, 2-hydroxyethyl methacrylate, aminopropylmethacrylate hydrochloride, and 3-methacryloylaminopropyl trimethylammonium chloride.

9. The implantable medical device of claim 1, the copolymer comprising at least about 80 mole percent N-isopropylacrylamide.

10. The implantable medical device of claim 1, the copolymer having a molecular weight of about of about 3,000 Da to about 17,000 Da.

11. A method of providing a biological material substrate comprising:
mixing a biological material with a copolymer formed from monomers comprising N-isopropylacrylamide or a thermally responsive derivative thereof and a second monomeric species including one or more of a heteroatom or a charged moiety;
increasing the temperature of the copolymer to form a hydrogel and allowing the biological material to undergo activity at the increased temperature;
lowering the temperature of the copolymer until the hydrogel dissolves; and
separating the biological material from the copolymer.

12. The method of claim 11, the biological material selected from the group consisting of cells, protein, and nucleic acid.

13. The method of claim 11, wherein the hydrogel is configured to simulate extracellular matrix.

14. The method of claim 11, wherein separating the biological material from the copolymer comprises size exclusion chromatography.

15. The method of claim 11, the copolymer comprising a substantially stable hydrogel from its lower critical solution temperature (LCST) to about 25 degrees Celsius above its LCST.

16. The method of claim 11, the second monomeric species including at least one of N,N-dimethylacrylamide, 2-hydroxyethyl methacrylate, aminopropylmethacrylate hydrochloride, and 3-methacryloylaminopropyl trimethylammonium chloride.

17. The method of claim 11, the copolymer comprising at least about 80 mole percent N-isopropylacrylamide.

18. The method of claim 11, the copolymer having a molecular weight of about of about 3,000 Da to about 17,000 Da.

19. A method of separating out components of a composition comprising
forming a hydrogel comprising raising the temperature of a copolymer formed from monomers comprising N-isopropylacrylamide or a thermally responsive derivative thereof and a second monomeric species including one or more of a heteroatom or a charged moiety to form a reagent mixture;
passing a mixture to be separated through the hydrogel;
segmenting the hydrogel into two or more parts;
selecting a segmented part of the hydrogel including a desired component of the mixture to be separated;
cooling the segmented part of the hydrogel until the copolymer dissolves; and
separating the dissolved copolymer from the desired component of the mixture to be separated.

20. The method of claim 19, further comprising adjusting the size of pores within the hydrogel by manipulating the temperature of the copolymer.

21. The method of claim 19, wherein passing the mixture to be separated through the hydrogel comprises creating a voltage differential between opposite sides of the hydrogel.
